(19) United States
(12) Patent Application Publication
(10) Pub. No.: US 2003/0026803 A1
(43) Pub. Date: Feb. 6, 2003

(54) Compositions for inhibiting macrophage activity

(75) Inventor: Neil Barclay, Oxford (GB)

Correspondence Address: PALMER & DODGE, LLP, KATHLEEN M. WILLIAMS, 111 HUNTINGTON AVENUE, BOSTON, MA, 02199, US

(73) Assignee: Medical Research Council

(21) Appl. No.: 10/178,089

(22) Filed: Jun. 24, 2002

Related U.S. Application Data
(63) Continuation of application No. PCT/GB00/04916, filed on Dec. 20, 2000.

(30) Foreign Application Priority Data
Dec. 24, 1999 (GB) 9930706.8

(51) Int. Cl. G01N 33/567; A61K 39/395

(52) U.S. Cl. 424/144.1; 435/7.21

(57) ABSTRACT

Use of an agent capable of inhibiting the interaction between SIRP1α and CD47, in the preparation of a composition for the inhibition of macrophage involvement in autoimmune disease. The invention further relates to a method for the treatment of an autoimmune disease with macrophage involvement, comprising administering to a mammal an effective amount of an agent which inhibits the interaction between CD47 and SIRP1α. The invention also relates to a method for identifying an agent capable of inhibiting the interaction between CD47 and SIRP1α, comprising the steps of exposing one or more test compounds to CD47 and/or SIRP1α, and monitoring the ability of the test compound to inhibit their interaction.

### FIELD OF THE INVENTION

This invention relates to compositions for inhibiting the involvement of macrophages in autoimmune disease, and the use of such compositions in the treatment of macrophage involvement in autoimmunity.

### BACKGROUND TO THE INVENTION

Cell proliferation, activation, differentiation, growth and survival are influenced by many extracellular factors. In the immune system and the nervous system such extracellular factors include chemokines, the extracellular matrix and neighbouring cells. The interaction of specific cell surface receptors with these factors triggers signalling cascades that stimulate the intracellular machinery to respond. The SIRP (signal regulatory protein) family has been recently described as a group of cell surface proteins that modulate cellular responses to growth factors and insulin (Kharitonenkov et al. (1997) Nature 386:181). The extracellular structure of SIRPs shows the presence of a domain common to adhesion molecules and immunoreceptors. The presence of immunoglobulin (Ig) domains suggests that SIRPs may interact with specific modulating ligands to achieve their regulatory effects.

Recently, CD47 (also known as integrin-associated protein or LAP) has been identified as the ligand for a murine neural adhesion molecule, a member of the SIRP family (Jiang et al., (1999) J. Biol. Chem. 274:559). Using an expression cloning strategy, Jiang et al. were able to show that CD47 is a major binding partner of P84. Moreover, Sarfati (WO99/40940) has speculated that ligands of CD47 may be useful for the treatment of autoimmune diseases. This speculation is based on results of experiments conducted on T cells and monocytes in vitro, which are asserted in WO99/40940 to support the hypotheses that (i) the inhibition by CD47 or CD47 ligand ligation of pro-inflammatory cytokine (including IL-2) production by monocytes and Il-2 responsiveness by T-cells could permit the downregulation of inflammatory responses on which new therapeutic strategies to chronic disorders could be based; (ii) the inhibition of Th-1 cell development and the production of unreactive or anergic cells may be the basis to develop novel strategies to induce allograft tolerance in organ or bone marrow transplantation; (iii) ligands of CD-47 may be developed for the treatment of CLL patients and (iv) the inhibition of IgE synthesis by CD47 ligation may be exploited to treat IgE-mediated allergic diseases.

Experimental allergic encephalomyelitis (EAE), a T-cell mediated autoimmune disease in rodents, is accepted as a relevant animal model for multiple sclerosis (MS) given the inflammatory changes observed in the CNS in these demyelinating disorders. EAE can be induced in mice or rats by injection of myelin basic protein (MBP) into the footpads in the presence of a strong adjuvant, such as Freund's complete adjuvant. After a period of approximately 12 days, susceptible animals develop a series of neurological symptoms caused by infiltration of immune system cells into the nervous system. The usual progress of the disease in rats, such as those of the Lewis strain, is such that the tail becomes limp, the hind limbs become paralysed and in the most severe cases the rats become incontinent. The disease is scored from 1 (limp tail) to 5 (incontinence). The disease usually resolves itself and rats recover about 18 days post injection.

### SUMMARY OF THE INVENTION

We have observed, as set forth below, that CD47 is a ligand for the SIRP1α OX41. OX41 was originally cloned using an antibody raised against macrophages (Robinson, et al., Immunology 57:239-247). An extensive study of the expression of OX41 has revealed that it is expressed on macrophages, monocytes, granulocytes, dendritic cells and neurons (Adams, et al., (1998) J. Immunol. 161:1853-9). Based on this observation, we have determined that substances which are capable of inhibiting the CD47-OX41 interaction on macrophages are capable of significantly limiting the severity of autoimmune diseases which display macrophage involvement.

Accordingly, in a first aspect of the present invention there is provided the use of an agent capable of inhibiting the interaction between macrophage SIRP1α and CD47, in the preparation of a composition for the inhibition of macrophage involvement in autoimmune disease.

Macrophage SIRP1α is a SIRP1α peptide present on macrophages, and is known as OX41 in rats. Stimulation of this peptide, with CD47/IAP, is postulated to result in activation of macrophages in autoimmune disease.

We have found that, by inhibiting the interaction of the macrophage SIRP1α with CD47, the severity of autoimmune conditions such as EAE can be significantly reduced or prevented. We accordingly postulate that SIRP1α plays an important role in macrophage activation during autoimmune disease.

As used herein, “SIRP1α”, “OX41” and “CD47” include variants of the naturally-occurring proteins, including mutants thereof which retain the characteristics of the natural proteins, such as mutants containing conservative amino acid substitutions, additions or deletions, or fragments thereof, such as single domains. For example, the extracellular domain of SIRP1α may be used in assays according to the invention, as it contains the regions of this protein responsible for binding to CD47. Similarly domains of CD47 may be isolated according to methods known in the art and used, alone or in the form of fusion proteins, in the present invention.

Preferably, the agent is a ligand of SIRP1α or CD47 which is capable of inhibiting the interaction between the two proteins. For example, the agent may be an inactive mimic or mutant of either CD47 or SIRP1α. Preferably, however, the agent is an antibody capable of binding to CD47 or SIRP1α and capable of inhibiting the CD47/SIRP1α interaction.

An “agent”, as referred to herein, may be substantially any molecule or process which is capable of achieving the required function, namely of inhibiting CD47/SIRP1α binding. Inhibitory molecules of a variety of types are known in the art, and can be used as a basis for the design of agents in accordance with the present invention. Preferred agents are antibodies or antibody fragments, which may be readily prepared and tested as described below, using techniques known in the art.

“Inhibiting” the CD47/SIRP1α interaction means that the functional relationship between the two molecules is altered such as to reduce or eliminate the activating effects on the macrophage by CD47. For example, the biological interaction between SIRP1α and CD47 may be reduced or altered. Alternatively, the binding of the two proteins may be inhibited or prevented.

“Inhibiting macrophage involvement” similarly refers to the reduction or prevention of the role and/or effect of macrophages in an autoimmune disease. For example, macrophage activation by CD47 may be prevented or reduced by the method of the invention.

“Autoimmune disease” is used in accordance with its ordinary signification in the art, namely to refer to a disease or component of a disease in which the immune system plays a damaging role by attacking “self” targets. Examples of autoimmune diseases include multiple sclerosis, arthritis and inflammatory bowel disease.

In a further embodiment, the invention relates to a method for the treatment of an autoimmune disease with macrophage involvement, comprising administering to a mammal an effective amount of an agent which inhibits the interaction between CD47 and SIRP1α.

The “treatment” of an autoimmune disease, as referred to herein, encompasses the alleviation or elimination of the symptoms and/or causes of an autoimmune disease as defined above.

In a further embodiment, the invention relates to a method for identifying an agent capable of inhibiting the interaction between CD47 and SIRP1α, comprising the steps of exposing one or more test compounds to CD 47 and/or SIRP1α, and monitoring the ability of the test compound to inhibit their interaction. The assay may be configured in a number of ways. For example, a first step in the assay may consist in determining whether the compound is capable of binding to CD47 or SIRP1α, i.e. is an SIRP1α or CD47 ligand. In a second step, CD47 or SIRP1α ligands may be assessed for their ability to inhibit SIRP1α/CD47 interaction. Alternatively, compounds may be tested directly for their ability to modulate the interaction between SIRP1α and CD47.

According to the present invention, therefore, SIRP1α or CD47 are used as targets to identify compounds, for example lead compounds for pharmaceuticals, which are capable of modulating the interaction between SIRP1α and CD47. Accordingly, the invention relates to an assay and provides a method for identifying a compound or compounds capable, directly or indirectly, of modulating the interaction of SIRP1α and CD47, comprising the steps of:

(a) incubating SIRP1α or CD47 with the compound or compounds to be assessed;

(b) identifying those compounds which bind to SIRP1α or CD47; and

(c) assessing those compounds which bind to SIRP1α or CD47 for their ability to modulate the interaction between SIRP1α and CD47. Advantageously, the interaction between SIRP1α and CD47 is assessed in a cell-based assay or in vivo.

### BRIEF DESCRIPTION OF THE FIGURES

FIG. 1. OX41-CD4d3+4 binds cells. A) recombinant chimeric OX41-CD4d3+4 protein. B) OX41-CD4d3+4 fluorescent beads used for ligand identification. C and D) OX41-CD4d3+4 but not control fluorescent beads bind to rat (C) thymocytes or (D) concanavalin A activated splenocytes. Preincubation of beads with OX41 mAb partially blocked binding of OX41-CD3+4 to cells relative to preincubation of beads with a control (OX21) mAb.

FIG. 2. OX101 mAb inhibits binding of OX41-CD4d3+4 beads to cells. Preincubation of (A) rat thymocytes or (B) concanavalin A activated splenocytes cells with OX101 mAb blocked binding of OX41-CD4d3+4 beads to the level seen with negative control beads. Preincubation of cells with another mAb reactive with both cell populations, CD48 mAb (OX45), did not block binding.

FIG. 3. OX101 mAb binds rat CD47. A) Example of protein eluted from OX101 mAb column in a fraction that was reactive with OX101 by Western blotting. Protein was analysed by SDS-PAGE under reducing conditions and visualised by silver staining. B) Western blot of protein from reactive fraction using OX101 mAb. C) Comparison of NH₂-terminal sequences of OX101 antigen and rat CD47.

FIG. 4. The interaction of hSIRP-Fc with immobilised hCD47-CD4d3+4 CD4 mAb OX68 was coupled to 3 flowcells of the BIAcore™. HCD47-CD4d3+4was bound to OX68 at equal levels in two flowcells. One of these flowcells was saturated with hCD47 mAb, BRIC126. SIRP-Fc (20 μg/ml) was passed over all 3 flowcells. SIRP-Fc bound to hCD47-CD4d3+4 (solid line) but when it was presaturated with hCD47 mAb binding was reduced (dashed line) to the level seen in the control flowcell (dotted line).

FIG. 5. Affinity and dissociation rate of soluble human CD47-CD4d3+4 binding to human SIRP-Fc. (A) Soluble monomeric hCD47-CD4d3+4 was injected at the indicated concentrations in 1 μM through flowcells immobilised hSIRP-Fc (6525 RU) (solid line), or as a negative control CTLA-4-Fc (6290 RU) (dotted line) at 37° C. (B) the difference between the response between the response at equilibrium in the hSIRP-Fc flowcell and the control flow cell is plotted against the hCD47-CD4d3+4 concentration. A k_{d}=8.5 μM and maximal binding of 1400 RU were calculated by non linear curve fitting of the Langmuir isotherm (line) to data (circles) from A with negative control subtracted. Inset shows Scatchard plot of data in B and calculated K_{d}=7.6 μM. (C) Soluble hCD47 (18.3 μM) was injected over immobilised hSIRP-Fc immobilised at high (6500 RU) (filled circles) and low (3000) levels (filled up triangles), and hCTLA-4-Fc (6290 RU) (filled down triangles). Values for k_{off}=1.6 s⁻¹ for hSIRP-Fc high, 2.1 s⁻¹ hSIRP-Fc low and 7.0 s⁻¹ for hCTLA-4-Fc were calculated by exponential decay curve fitting (line) to dissociation data (symbols).

FIG. 6. The NH2-terminal V domain of SIRP binds CD47. Soluble rat CD4 and hCD47-CD4d3+4 were injected into flowcells with immobilised V-SIRP-Cd4d3+4 (solid line) or control rat CD4 (dashed line).

FIG. 7. Schematic representation of rat OX41/SIRP and human SIRPα and CD47. Species differences in potential N-linked sites are shown.

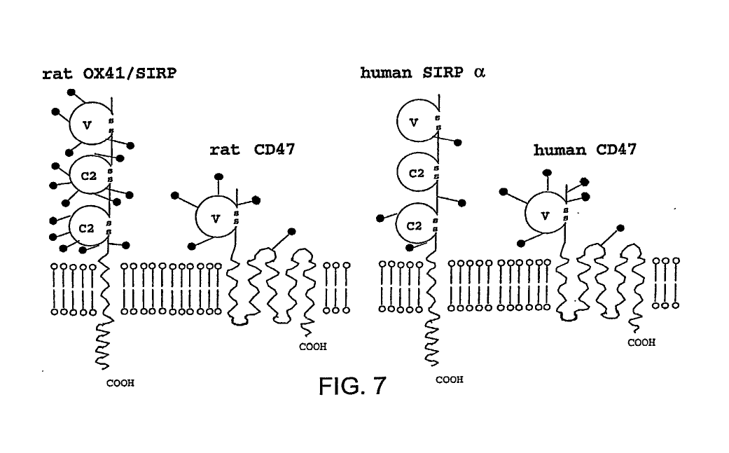

### DETAILED DESCRIPTION OF THE INVENTION

1. Agents which inhibit the SIRP1α/CD47 Interaction

1a. CD47/SIRP1α Binding Compounds

According to a first embodiment of this aspect invention, the assay is configured to detect polypeptides which bind directly to CD47 or SIRP1α.

The invention therefore provides a method for identifying a modulator of the CD47-SIRP1α interaction, comprising the steps of:

(a) incubating SIRP1α or CD47 with the compound or compounds to be assessed; and

(b) identifying those compounds which bind to SIRP1α or CD47.

Preferably, the method further comprises the step of:

(c) assessing the compounds which bind to SIRP1α or CD47 for the ability to modulate the SIRP1α-CD47 interaction in a cell-based assay.

Binding to SIRP1α or CD47 may be assessed by any technique known to those skilled in the art. Examples of suitable assays include the two hybrid assay system, which measures interactions in vivo, affinity chromatography assays, for example involving binding to polypeptides immobilised on a column, fluorescence assays in which binding of the compound(s) and SIRP1α or CD47 is associated with a change in fluorescence of one or both partners in a binding pair, surface plasmon resonance analysis, and the like. Preferred are assays performed in vivo in cells, such as the two-hybrid assay. Such assays may be configured to assess CD47-SIRP1α interaction simultaneously, since the binding pair used in the two-hybrid assay may be based on CD47 and SIRP1α.

Therefore, the invention also provides a method for identifying a lead compound for a pharmaceutical useful in the treatment of macrophage involvement in autoimmune diseases, comprising incubating a compound or compounds to be tested with SIRP1α and CD47, under conditions in which, but for the presence of the compound or compounds to be tested, CD47 associates with SIRP1α with a reference affinity;

determining the binding affinity of CD47 for SIRP1α in the presence of the compound or compounds to be tested; and

selecting those compounds which modulate the binding affinity of CD47 for SIRP1α with respect to the reference binding affinity.

Such methods, although they determine the ability of the test compound to modulate CD47/SIRP1α interaction, do not assess the ability of the compound to regulate the interaction at a functional level. Such assays are preferably based on systems in which activation of SIRP1α is observable.

The N-terminal domain of SIRP1α is responsible for binding with CD47. Accordingly, agents which interact with SIRP1α preferably interact with the N-terminal thereof.

1b. Compounds which Modulate the Functional CD47-SIRP1α Interaction

In a further embodiment, the invention may be configured to detect functional interactions between a CD47 and SIRP1α. Such interactions will occur either at the level of the regulation of SIRP1α, by determining whether this signalling molecule is itself activated in response to CD47 in the presence of the compound or compounds to be tested, or at the level of the modulation of the biological effect of CD47 on macrophages or other cells which express SIRP1α. As used herein, “activation” and “inactivation” include modulation of the activity, enzymatic or otherwise, of a compound.

Assays which detect modulation of the functional interaction between CD47 and SIRP1α are preferably cell-based assays. For example, they may be based on an assessment of signalling activity by SIRP1α, which is indicative of the degree of activation, resulting from the CD47-SIRP1α interaction.

In preferred embodiments, a nucleic acid encoding SIRP1α is ligated into a vector, and introduced into suitable host cells to produce transformed cell lines that express SIRP1α . The resulting cell lines can then be produced for reproducible qualitative and/or quantitative analysis of the effect(s) of potential compounds affecting the CD47-SIRP1α interaction. Alternatively, cells which naturally express SIRP1α, such as macrophages, may be used for the same purposes.

Thus SIRP1α expressing cells may be employed for the identification of compounds, particularly low molecular weight compounds, which for example modulate the binding of CD47 to SIRP1α.

Cell-based screening assays can be designed by constructing cell lines in which the expression of a reporter protein, i.e. an easily assayable protein, such as β-galactosidase, chloramphenicol acetyltransferase (CAT) or luciferase, is dependent on the activation of SIRP1α by CD47. For example, a reporter gene encoding one of the above polypeptides may be placed under the control of a SIRP-responsive promoter which is specifically activated by the SIRP1α pathway. Such an assay enables the detection of compounds that directly modulate the CD47-SIRP1α interaction, such as compounds that prevent CD47 binding to SIRP1α.

Alternative assay formats include assays which directly assess macrophage activation in a biological system. The activation of SIRP1α by CD47 results n the involvement of macrophages in autoimmune diseases.

2. Agents

In a still further aspect, the invention relates to an agent or agents identifiable by an assay method as defined in the previous aspect of the invention. Accordingly, there is provided the use of an agent identifiable by an assay as described herein, for the modulation of the activity of macrophages in autoimmune disease.

Compounds which influence the CD47-SIR1α interaction may be of almost any general description, including low molecular weight compounds, including organic compounds which may be linear, cyclic, polycyclic or a combination thereof, peptides, polypeptides including antibodies, or proteins. In general, as used herein, “peptides”, “polypeptides” and “proteins” are considered equivalent.

2a. Antibodies

Antibodies, as used herein, refers to complete antibodies or antibody fragments capable of binding to a selected target, and including Fv, ScFv, Fab′ and F(ab′)₂, monoclonal and polyclonal antibodies, engineered antibodies including chimeric, CDR-grafted and humanised antibodies, and artificially selected antibodies produced using phage display or alternative techniques. Small fragments, such Fv and ScFv, possess advantageous properties for diagnostic and therapeutic applications on account of their small size and consequent superior tissue distribution.

The antibodies according to the invention are especially indicated for diagnostic and therapeutic applications. Accordingly, they may be altered antibodies comprising an effector protein such as a toxin or a label. Especially preferred are labels which allow the imaging of the distribution of the antibody in vivo. Such labels may be radioactive labels or radioopaque labels, such as metal particles, which are readily visualisable within the body of a patient. Moreover, the may be fluorescent labels or other labels which are visualisable on tissue samples removed from patients.

Recombinant DNA technology may be used to improve the antibodies of the invention. Thus, chimeric antibodies may be constructed in order to decrease the immunogenicity thereof in diagnostic or therapeutic applications. Moreover, immunogenicity may be minimised by humanising the antibodies by CDR grafting [see European Patent Application 0 239 400 (Winter)] and, optionally, framework modification [see 0 239 400 (Winter) and the art reviewed in international patent application WO 90/07861 (Protein Design Labs)].

Antibodies according to the invention may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Therefore, the present invention includes a process for the production of an antibody according to the invention comprising culturing a host, e.g. E. coli or a mammalian cell, which has been transformed with a hybrid vector comprising an expression cassette comprising a promoter operably linked to a first DNA sequence encoding a signal peptide linked in the proper reading frame to a second DNA sequence encoding said protein, and isolating said protein.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by immunofluorescent staining of cells expressing SIRP1α or CD47, by immunoblotting, by an enzyme imunoassay, e.g. a sandwich assay or a dot-assay, or a radioimmunoassay.

For isolation of the antibodies, the immunoglobulins in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with SIRP1α, CD47 or with Protein-A.

The invention further concerns hybridoma cells secreting the monoclonal antibodies of the invention. The preferred hybridoma cells of the invention are genetically stable, secrete monoclonal antibodies of the invention of the desired specificity and can be activated from deep-frozen cultures by thawing and recloning.

The invention also concerns a process for the preparation of a hybridoma cell line secreting monoclonal antibodies directed to SIRP1α or CD47, characterised in that a suitable mammal, for example a Balb/c mouse, is immunised with purified SIRP1α or CD47, an antigenic carrier containing purified SIRP1α or CD47or with cells bearing SIRP1α or CD47, antibody-producing cells of the immunised mammal are fused with cells of a suitable myeloma cell line, the hybrid cells obtained in the fusion are cloned, and cell clones secreting the desired antibodies are selected. For example spleen cells of Balb/c mice immunised with cells bearing SIRP1α or CD47 are filsed with cells of the myeloma cell line PAI or the myeloma cell line Sp2/0-Ag14, the obtained hybrid cells are screened for secretion of the desired antibodies, and positive hybridoma cells are cloned.

Preferred is a process for the preparation of a hybridoma cell line, characterised in that Balb/c mice are immunised by injecting subcutaneously and/or intraperitoneally between 10 and 10⁷ and 10⁸ cells of human tumour origin which express SIRP1α or CD47 containing a suitable adjuvant several times, e.g. four to six times, over several months, e.g. between two and four months, and spleen cells from the immunised mice are taken two to four days after the last injection and fused with cells of the myeloma cell line PAI in the presence of a fusion promoter, preferably polyethylene glycol. Preferably the myeloma cells are fused with a three- to twentyfold excess of spleen cells from the immunised mice in a solution containing about 30% to about 50% polyethylene glycol of a molecular weight around 4000. After the fusion the cells are expanded in suitable culture media as described hereinbefore, supplemented with a selection medium, for example HAT medium, at regular intervals in order to prevent normal myeloma cells from overgrowing the desired hybridoma cells.

The invention also concerns recombinant DNAs comprising an insert coding for a heavy chain variable domain and/or for a light chain variable domain of antibodies directed to SIRP1α or CD47 as described hereinbefore. By definition such DNAs comprise coding single stranded DNAs, double stranded DNAs consisting of said coding DNAs and of complementary DNAs thereto, or these complementary (single stranded) DNAs themselves.

Furthermore, DNA encoding a heavy chain variable domain and/or for a light chain variable domain of antibodies directed to SIRP1α or CD47 can be enzymatically or chemically synthesised DNA having the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant DNA is also intended to be a silent mutant wherein one or more nucleotides are replaced by other nucleotides with the new codons coding for the same amino acid(s). Such a mutant sequence is also a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly E. coli, to obtain an optimal expression of the heavy chain murine variable domain and/or a light chain murine variable domain.

The term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

The invention therefore also concerns recombinant DNAs comprising an insert coding for a heavy chain murine variable domain of an antibody directed to SIRP1α or CD47 fused to a human constant domain g, for example γ1, γ2, γ3 or γ4, preferably γ1 or γ4. Likewise the invention concerns recombinant DNAs comprising an insert coding for a light chain murine variable domain of an antibody directed to SIRP1α or CD47 fused to a human constant domain κ or λ, preferably κ.

In another embodiment the invention pertains to recombinant DNAs coding for a recombinant polypeptide wherein the heavy chain variable domain and the light chain variable domain are linked by way of a spacer group, optionally comprising a signal sequence facilitating the processing of the antibody in the host cell and/or a DNA coding for a peptide facilitating the purification of the antibody and/or a cleavage site and/or a peptide spacer and/or an effector molecule.

The DNA coding for an effector molecule is intended to be a DNA coding for the effector molecules useful in diagnostic or therapeutic applications. Thus, effector molecules which are toxins or enzymes, especially enzymes capable of catalysing the activation of prodrugs, are particularly indicated. The DNA encoding such an effector molecule has the sequence of a naturally occurring enzyme or toxin encoding DNA, or a mutant thereof, and can be prepared by methods well known in the art.

Antibodies and antibody fragments according to the invention are useful in diagnosis and therapy. Accordingly, the invention provides a composition for therapy or diagnosis comprising an antibody according to the invention.

In the case of a diagnostic composition, the antibody is preferably provided together with means for detecting the antibody, which may be enzymatic, fluorescent, radioisotopic or other means. The antibody and the detection means may be provided for simultaneous, simultaneous separate or sequential use, in a diagnostic kit intended for diagnosis.

2b. Peptides

Peptides according to the present invention are usefully derived from SIRP1α, CD47 or another polypeptide involved in the functional SIRP1α-CD47 interaction. Preferably, the peptides are derived from the domains in SIRP1α or CD47 which are responsible for SIRP1α-CD47 interaction. For example, Thornberry et al, (1994) Biochemistry 33:3934-3940 and Milligan et al., (1995) Neuron 15:385-393 describe the use of modified tetrapeptides to inhibit ICE protease. In an analogous fashion, peptides derived from SIRP1α or CD47 may be modified, for example with an aldehyde group, chloromethylketone, (acyloxy) methyl ketone or CH₂OC(O)-DCB group to inhibit the SIRP1α-CD47 interaction.

In order to facilitate delivery of peptide compounds to cells, peptides may be modified in order to improve their ability to cross a cell membrane. For example, U.S. Pat. No. 5,149,782 discloses the use of fusogenic peptides, ion-channel forming peptides, membrane peptides, long-chain fatty acids and other membrane blending agents to increase protein transport across the cell membrane. These and other methods are also described in WO 97/37016 and U.S. Pat. No. 5,108,921, incorporated herein by reference.

Many compounds according to the present invention may be lead compounds useful for drug development. Useful lead compounds are especially antibodies and peptides, and particularly intracellular antibodies expressed within the cell in a gene therapy context, which may be used as models for the development of peptide or low molecular weight therapeutics. In a preferred aspect of the invention, lead compounds and SIRP1α or CD47 may be co-crystallised in order to facilitate the design of suitable low molecular weight compounds which mimic the interaction observed with the lead compound.

Crystallisation involves the preparation of a crystallisation buffer, for example by mixing a solution of the peptide or peptide complex with a “reservoir buffer”, preferably in a 1:1 ratio, with a lower concentration of the precipitating agent necessary for crystal formation. For crystal formation, the concentration of the precipitating agent is increased, for example by addition of precipitating agent, for example by titration, or by allowing the concentration of precipitating agent to balance by diffusion between the crystallisation buffer and a reservoir buffer. Under suitable conditions such diffusion of precipitating agent occurs along the gradient of precipitating agent, for example from the reservoir buffer having a higher concentration of precipitating agent into the crystallisation buffer having a lower concentration of precipitating agent. Diffusion may be achieved for example by vapour diffusion techniques allowing diffusion in the common gas phase. Known techniques are, for example, vapour diffusion methods, such as the “hanging drop” or the “sitting drop” method. In the vapour diffusion method a drop of crystallisation buffer containing the protein is hanging above or sitting beside a much larger pool of reservoir buffer. Alternatively, the balancing of the precipitating agent can be achieved through a semipermeable membrane that separates the crystallisation buffer from the reservoir buffer and prevents dilution of the protein into the reservoir buffer.

In the crystallisation buffer the peptide or peptide/binding partner complex preferably has a concentration of up to 30 mg/ml, preferably from about 2 mg/l to about 4 mg/ml.

Formation of crystals can be achieved under various conditions which are essentially determined by the following parameters: pH, presence of salts and additives, precipitating agent, protein concentration and temperature. The pH may range from about 4.0 to 9.0. The concentration and type of buffer is rather unimportant, and therefore variable, e.g. in dependence with the desired pH. Suitable buffer systems include phosphate, acetate, citrate, Tris, MES and HEPES buffers. Useful salts and additives include e.g. chlorides, sulphates and other salts known to those skilled in the art. The buffer contains a precipitating agent selected from the group consisting of a water miscible organic solvent, preferably polyethylene glycol having a molecular weight of between 100 and 20000, preferentially between 4000 and 10000, or a suitable salt, such as a sulphates, particularly ammonium sulphate, a chloride, a citrate or a tartarate.

A crystal of a peptide or peptide/binding partner complex according to the invention may be chemically modified, e.g. by heavy atom derivatization. Briefly, such derivatization is achievable by soaking a crystal in a solution containing heavy metal atom salts, or a organometallic compounds, e.g. lead chloride, gold thiomalate, thimerosal or uranyl acetate, which is capable of diffusing through the crystal and binding to the surface of the protein. The location(s) of the bound heavy metal atom(s) can be determined by X-ray diffraction analysis of the soaked crystal, which information may be used e.g. to construct a three-dimensional model of the peptide.

A three-dimensional model is obtainable, for example, from a heavy atom derivative of a crystal and/or from all or part of the structural data provided by the crystallisation. Preferably building of such model involves homology modelling and/or molecular replacement.

The preliminary homology model can be created by a combination of sequence alignment with any SIRP the structure of which is known, secondary structure prediction and screening of structural libraries.

Computational software may also be used to predict the secondary structure of the peptide or peptide complex. The peptide sequence may be incorporated into the SIRP1α or CD47 structure. Structural incoherences, e.g. structural fragments around insertions/deletions can be modelled by screening a structural library for peptides of the desired length and with a suitable conformation. For prediction of the side chain conformation, a side chain rotamer library may be employed.

The final homology model is used to solve the crystal structure of the peptide by molecular replacement using suitable computer software. The homology model is positioned according to the results of molecular replacement, and subjected to further refinement comprising molecular dynamics calculations and modelling of the inhibitor used for crystallisation into the electron density.

3. Pharmaceutical Compositions

In a preferred embodiment, there is provided a pharmaceutical composition comprising a compound or compounds identifiable by an assay method as defined in the previous aspect of the invention.

A pharmaceutical composition according to the invention is a composition of matter comprising a compound or compounds capable of modulating SIRP1α-activating activity of CD47 as an active ingredient. The active ingredients of a pharmaceutical composition comprising the active ingredient according to the invention are contemplated to exhibit excellent therapeutic activity, for example, in the treatment of tumours or other diseases associated with cell proliferation, infections and inflammatory conditions, when administered in amount which depends on the particular case. Dosage regima may be adjusted to provide the optimum therapeutic response. For example, several divided doses may be administered daily or the dose may be proportionally reduced as indicated by the exigencies of the therapeutic situation.

The active ingredient may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

In order to administer the active ingredient by other than parenteral administration, it will be coated by, or administered with, a material to prevent its inactivation. For example, the active ingredient may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active ingredient may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active ingredient in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredient is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active ingredient may be incorporated with excipients and used in the form of ingestible tablets, buccal tablets, troches, capsules, elixirs, suspensions, syrups, wafers, and the like. The amount of active ingredient in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active ingredient, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the active ingredient of the invention as hereinbefore defined for use in the treatment of disease. Consequently there is provided the use of an active ingredient of the invention for the manufacture of a medicament for the treatment of disease associated with NFκB induction or repression.

Moreover, there is provided a method for treating a condition associated with NFκB induction or repression, comprising administering to a subject a therapeutically effective amount of a compound or compounds identifiable using an assay method as described above.

The invention is further described, for the purpose of illustration only, in the following examples.

### Experimental Procedures

Animals and Monoclonal Antibodies

The following monoclonal antibodies were used; anti-rat SIRP mAb SIRP1α (Adams, et al., (1998) J. Immunol. 161:1853-9; Robinson et al., (1986) Immunology 57:239-47) and anti-rat CD2 mAb OX 34 and anti rat CD4 domains 3+4 mAb OX68 which are referenced in the European Collection of Animal Cell Cultures (Porton Down, Salisbury, G.B.). The OX101 (IgG1) antibody was selected from a panel of antibodies raised in Balb/C mice against biotinylated rat thymocytes. hCD47 mAbs were BRIC 126 (Serotec, Kidlington, UK) and mAb 1796 (Chemicon, Temecula, Calif., USA).

Thymocytes and other cell preparations were prepared from Oxford albino (AO) rats between 4-8 weeks of age of either sex. Activated splenocytes were prepared by culturing 3.5×10⁶ cells/ml in RPMI with 5% foetal calf serum and 5 μg/ml concanavalin A for 48-72 hours.

Soluble Fusion Protein Constructs and Full Length Constructs

Construction of rat CD4 domain 3+4 biotin chimeric proteins has been described previously (Brown et al., (1998) J. Exp. Med. 188:2083-90; Brown and Barclay, (1994) Prot. Eng. 7:515-21). SIRP1α cDNA (Adams, et al., (1998) J. Immunol. 161:1853-9) was excised from pCDM8 by digestion with Pst I and Hind III and subcloned into Bluescript vector (Stratagene GMVH, Heidelberg, Germany). A Sal I site was introduced at the end of the extracellular region by single strand mutagenesis (Biorad, Hemel Hempsted, Herts. UK). An Xba I-Sal I fragment encoding the extracellular region of SIRP1α was inserted into pEF-BOS-XB (Brown et al., (1998) J. Exp. Med. 188:2083-90) upstream of CD4 domains 3+4-biotin (Brown et al., (1998) J. Exp. Med. 188:2083-90; Brown and Barclay, (1994) Prot. Eng. 7:515-21). The resulting junction with CD4d3 was gtcaccaagggtcgacatccatcaeg (VTQGSTSIT). The extracellular domain of human CD47 was amplified from plasmid DNA (donated by Dr Ian Campbell, Southampton, UK) by PCR and inserted into the CD4d3+4-biotin pEF-BOS-XB vector with the following junction cgtgttgtttcatggtcgatccatc (RVVSWSTSI—human CD47). The Xba I-Bam HI fragment encoding hCD47-CD4d3+4 was transferred to pEE14(18) for production of a stable line. The single SIRP V domain was amplified by PCR with primers ataaagcgtctagagcggatatgtggcccctg (sense) and catttggagtcgaccatgaaacaacac (antisense) to construct a CD4d3+4 fusion protein. SIRP-1α was amplified by PCR from a HT1080 fibrosarcoma cDNA library with the primers gcaagcttatggagcccgccggc (sense) and cagattcgtcccattcacttcc (antisense). The PCR product was digested with Hind III EcoR I and inserted into the mammalian expression vector pCDNA 3.0 (Invitrogen, Groningen, Netherlands). The recombinant fusion protein coupling SIRP to human IgG Fc was made by amplifying extracellular domains of SIRP-1α with Hind III, Not I compatible ends and inserting the digested PCR product into pIg PLUS (R&D Systems, Oxford, UK). The junction at the 3′ end of the extracellular region corresponds to cactggatctaatgaacgg (TGSNER).

Cell Culture Expression

Fusion protein constructs were transiently expressed in COS-1 cells (Imperial Cancer Research Fund) (20 μg DNA/10⁷ cells) using DEAE-dextran (19) or in 293T cells using calcium phosphate for biotinylation experiments. COS cells transfected with constructs for cell surface expression were passaged 24 hours post transfection and used in flow cytometry 72-96 hours post transfection. Expression of soluble CD3+4 proteins was detected by inhibition ELISA (Brown and Barclay, (1994) Prot. Eng. 7:515-21). Supernatants were dialysed against 10 mM Tris-HCl, pH8.0 and concentrated using 10,000 MW cut off centricons (Amicon, Beverley, Mass., USA). Enzymatic biotinylation was performed using 1 μg (5,000 units) of the recombinant E. coli BirA enzyme (Avidity, Denver, Colo., USA) as recommended by the manufacturers. Excess biotin was removed by dialysis against PBS. For use in BIAcore experiments, HCD47-CD4d3+4 was purified from a stable Chinese hamster ovary cell line produced using the glutamine synthetase system (Davis et al., (1990) J. Biol. Chem. 265:10410-8) by affinity chromatography with OX68 mAb. The extinction coefficient (1.0 cm²/mg) was determined experimentally from amino acid composition and concentration was measured by absorption at 280 nm. Subsequent gel filtration using Superdex 75 (Pharmacia Biotech Ltd) was carried out and fractions used in BIAcore analysis without further concentration.

Microsphere (Bead) Binding Assays

Binding experiments were carried out and analysed by flow cytometry as described (Brown et al., (1995) Eur. J. Immunol. 25: 3222-8; Brown et al., (1998) J. Exp. Med. 188:2083-90). Typically, 5×10⁵ thymocytes were plated in flat bottomed microtitre plates and where appropriate incubated with 50 μl hybridoma tissue-culture supernatant for 1 hour at 4° C. Fluorescent streptavidin coated microspheres 1.5×10⁸ 15 μl (Spherotech, Libertyville, Ill., USA cat no VFP-0552-5) were mixed with recombinant OX 41-CD4d3+4 protein (2 μg/sample) for 1 hour at 4° C. Beads coated with rat CD4d3+4, rat CD5-CD4d3+4 or rat CD48-CD4d3+4 binding to cells precoated with a blocking CD2 mAb were used as superimposable negative controls (Brown et al., (1998) J. Exp. Med. 188:2083-90). Unbound protein was removed by washing the beads in 0.2% BSA/PBS. Coated beads were sonicated for 1 minute and then incubated with the cells for 1 hour at 4° C. as described (Brown et al., (1998) J. Exp. Med. 188:2083-90).

Affinity Purifcation of OX 101 Antigen

Thymocyte membranes were prepared as described (Williams et al., (1986) in Handbook of Experimental Immunology, Vol. 1, 4^{th} Ed., pp. 22.1-22.24, Blackwell Scientific, Oxford) and solubilised in 2% Na deoxycholate, 10 mM Tris-HCl, 0.02% NaN₃ buffer pH 8 containing 2.5 mM iodoacetamide, 0.2 mM PMSF. The solubilised membrane preparation was applied to OX101 mAb coupled to CNBr-Sepharose (Sigma, Poole, Dorset, UK), washed until A₂₈₀ nm dropped to <0.02 and OX101 antigen was eluted with a high pH buffer (0.05M diethylamine-HC1, 0.5% Na deoxycholate, 0.02% NAN₃ pH 11.5).

Antigen Analysis

Eluted fractions were electrophoresed through a 10% SDS-PAGE gel under reducing conditions and immunoreactivity with the OX101 mAb was examined by subsequent Western blotting. Reactive bands were visualised with goat anti-mouse antibodies conjugated to horseradish peroxidase (Sigma, Poole, Dorset, UK) and a chemiluminescent peroxidase substrate (ECL, Amersham, Bucks. UK). Reactive fractions were precipitated by adding 1/10 volume of 72% thrichloroacetic acid, incubating at room temperature for 5 minutes and centrifuging at 10 000 g for 10 minutes. The pelleted protein was analysed by SDS PAGE and silver staining. The stained band corresponding to the immunoreactive region on OX101 Western blots was excised and used for N-terminal sequencing by Edman degradation using an Applied Biosystems Procise 494A protein sequencer (Perkin-Elmer Ltd., UK).

BIAcore Analysis

BIAcore analysis was carried out using a BIAcore™ 2000 biosensor instrument (Biacore AB) as previously described (Brown et al., (1998) J. Exp. Med. 188:2083-90, van der Merwe, et al., (1994) Biochemistry 33:101489-60) using CM5 research grade chips. The hSIRP-Fc and hCTLA-4Fc proteins were immunobilised directly via amine coupling at 50 μg/ml in 10 mM Na acetate pH 4.5. equilibrium affinity and kinetic measurements were carried out at 37° C. using short injection times of 3 s (5 μl at 100 μl/min) to minimise the contribution of any aggregated material. For equilibrium binding increasing and decreasing concentrations of monomeric hCD47-CD4d3+4 were passed over hSIRP-Fc (6500 RU). For kinetic measurements hCD47-CD4d3+4 (18.30 μM) was passed over hSIRP-Fc immobilised at 6500 RU and 3000 RU and a control, CTLA4-Fc (6300 RU). CD47mAb blocking and V-SIRP-CD4d3+4 binding experiments were carried out at 10 and 5 μl/min respectively at 25° C.

### EXAMPLE 1

OX41 is a Ligand for CD47

OX41-CD4d3+4 Microspheres Bind Thymocytes and Concanavalin A Treated Splenocytes

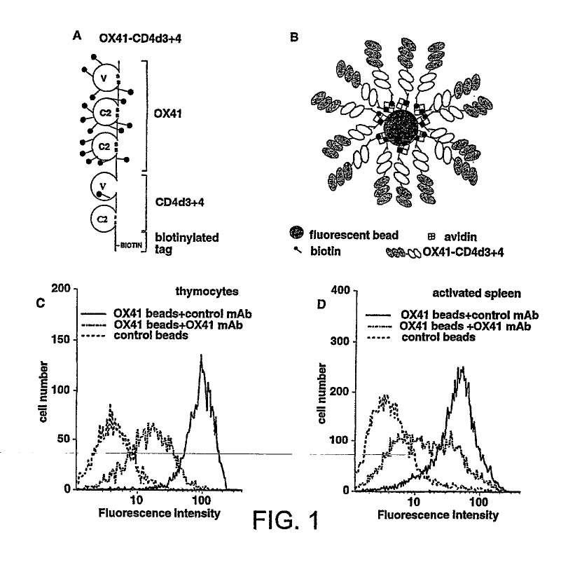

To identify a ligand for the rat OX41 antigen a recombinant fusion protein was made by expressing the OX41 extracellular domain upstream of domains 3 and 4 of rat CD4 and a short peptide recognised by the E. coli biotinylating enzyme BirA (Schatz (1993) Biotechnology 11:1138-43) (FIG. 1A). Biotinylated OX41-CD4d3+4 protein bound the OX41mAb consistent with it being correctly folded.

Fluorescent Sphero™ avidin coated microspheres labelled with biotinylated OX41-CD4d3+4 (FIG. 1B) were used to identify cell types bearing SIRP ligands. These OX41-CD4d3+4 beads bound to rat thymocytes and concanavalin A activated rat spleen cells as assessed by flow cytometry (FIGS. 1C and D). Binding was comparable to that observed with a previously characterised interaction between rat CD48-CD4d3+4 coated beads binding to their ligand CD2 on these cells (Brown et al., (1995) Eur. J. Immunol. 25:3222-8). These interactions were judged to be specified as they were partially blocked by preincubating OX41-CD4d3+4 beads with OX41mAb but not by irrelevant control mAbs, OX21 or an IgG2a isotype matched control OX34 mAb.

OX101 mAb specifically blocks binding of OX41-CD4d3+4 to cells

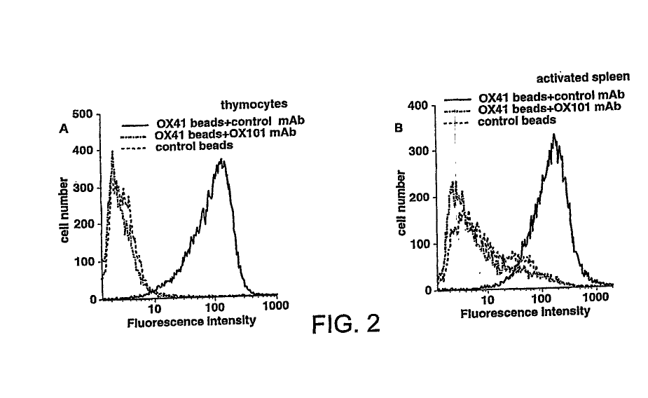

Cell binding data with OX41-CD4d3+4 beads suggested that rat thymocytes express a ligand for OX41. In order to identify the ligand a panel of approximately 50 mouse mAbs that had been raised against rat thymocytes was screened for blocking activity in the OX41 bead binding assay. One antibody, OX101, gave clear inhibition of the interaction of OX41-CD4d3+4 labelled beads with thymocytes (FIG. 2A). Other mAbs reacting with thymocytes gave no inhibition as shown for CD48 mAb (FIG. 2A) indicating that this was likely to be a specific effect. OX101 also blocked the binding of OX41-CD4d3+4 beads concanavalin A treated splenocytes (FIG. 2B). Flow cytometry showed that OX101 labelled the majority of thymocytes, splenocytes, cervical lymph node cells and peritoneal cells indicating that it was present in the majority of lymphocytes and macrophages (not shown). In addition, flow cytometry revealed that a crude preparation of brain tissue also contained OX101 antigen (not shown).

Isolation and Identification of OX101 Antigen as Rat CD47

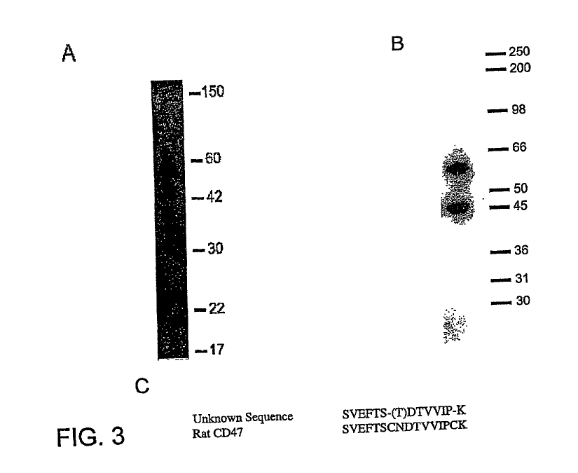

The OX101 antigen was purified using an OX101 mAb affinity column. Specifically bound protein was eluted from the OX101 mAb column at high pH (FIG. 3A). Fractions were screened for the OX101 antigen by Western blotting with the OX101mAb (FIG. 3B). Two reactive bands corresponding to molecular weights of approximately 60 and 45 kD were observed. Fractions containing reactive protein were reprecipitated and separated by SDS PAGE. In the 45-60kD region the reprecipitated material ran as one diffuse band. This region was excised and sequenced (FIG. 3C). The single sequence obtained was virtually identical to the NH₂-terminal sequence of rat CD47 also known as integrin associated protein (Genbank accession numbers D87659, AF17437). The sequencing result is consistent with the two bands observed by Western blotting arising from different glycosylated forms or the lower band being due to degradation at the C-terminus.

SIRP/OX41 Binds Directly to CD47

To establish the significance of the OX41-CD4d3+4 cell binding data in rat and identification of the OX101 antigen as CD47, we asked whether there was a direct interaction between OX41/SIRP and CD47 proteins, and whether this interaction was conserved in human. Purified recombinant chimeric proteins comprising of the extracellular region of human SIRPα and the Fc region from IgG (hSIRP-Fc) of the single extracellular IgSF domain of human CD47 with CD4d3+4 (hCD47-CD4d3+4) were tested for binding using a BIAcore.

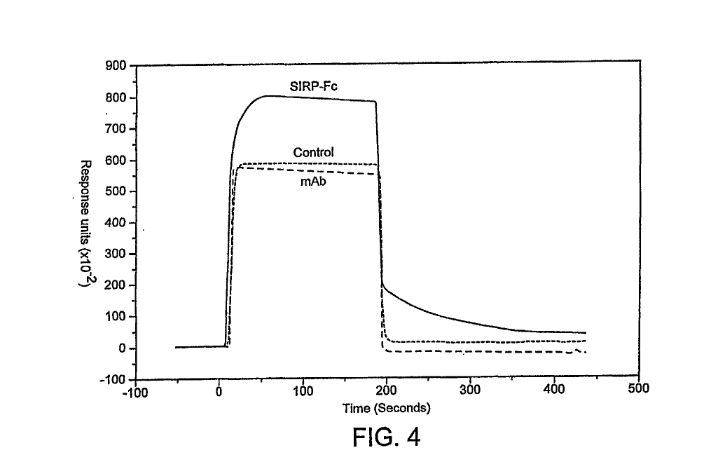

HCD47-CD4d3+4 was immobilised in two flowcells via a CD4 mAb, OX68. One flowcell was saturated with hCD47 mAb. Dimeric hSIRP-Fc was passed over both flowcells and a control flowcell (FIG. 4) hSIRP-Fc bound specifically to hCD47-CD4d3+4. The specificity of the interaction was confirmed by showing that preincubation with hCd47 mAbs, BRIC126 (FIG. 4) and 1796 (data not shown), blocked the biding of HSIRP-Fc to the level of the control flowcell.

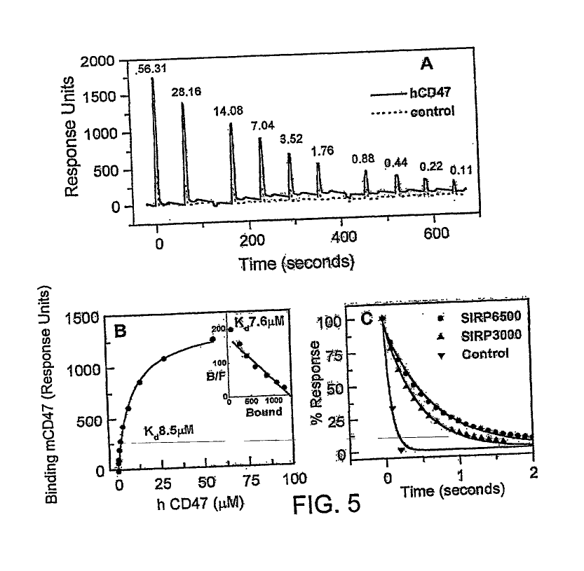

To measure the affinity of the interaction hSIRP-Fc was directly immobilised and monomeric hCD47-CD4d3+4 passed over it. As shown in FIG. 5A hCD47-CD4d3+4 bound specifically to hSIRP-Fc in a dose dependent manner. Non linear curve fitting of the data collected at 37° C. produced a k_{d}=8.5 μM (FIG. 5B). Scatchard analysis gave a similar result (FIG. 5B inset.) Eight determinations of the kd over two levels of immobilised SIRP-Fc fell within the range 2-9 μM. The apparent rate constants were calculated as k_{off}=1.3 s⁻¹ for the higher level of hSIRP-Fc and k_{off}=2.1 s⁻¹ for the lower level (FIG. 5C). This increase in the apparent k_{off} most likely reflects the existence of rebinding effects after dissociation which result in underestimation of the true k_{off}. Thus the true k_{off} is ≧2.1 s⁻¹. From the dissociation rate constant koff and the kd the association rate constant k_{off} and the k_{d} the association rate constant was calculated to be 1.7×10⁻⁵M⁻¹s⁻¹. Although it is difficult to measure rapid association rates Kₒₙ can be estimated by curve fitting to the association phase of the binding data shown in FIG. 5C. For hCD47-CD4d3+4 (18.3 μM) binding to SIRP (6500 RU) the Kₒₙ was measured as 1.9×10⁻⁵M⁻¹s⁻¹.

The NH₂-terminal V-like Domain of SIRP/OX41 Binds Directly to CD47

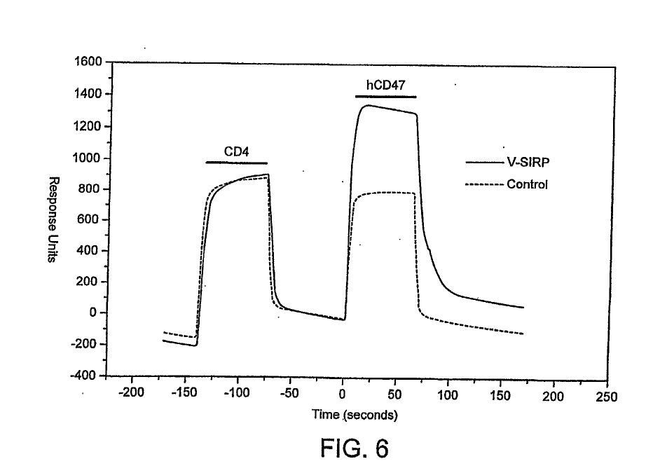

To localise the CD47 binding site on SIRP a construct containing only the NH₂-terminal V-like domain of hSIRP in frame with CD4d3+4 was made. V-SIRP-CD4d3+4 was immobilised in one flowcell via a CD4 mAb (OX68). The free OX68 in this flowcell and all the OX68 in the control flowcell was blocked with soluble rat CD4. Blocking was verified by repeating the injection of soluble rat CD4 (FIG. 6). Soluble hCD47-CD4d3+4 was then injected over both the flowcells. There was no difference in CD4 binding between the two flowcells whereas there was specific binding of hCD47-CD4d3+4 to the single V-like domain of SIRP (FIG. 6).

### EXAMPLE 2

An Anti-CD47 Antibody Reduces the Severity of EAE In Vivo.

Lewis rats were injected with myelin basic protein (MBP) into the footpads together with Freund's complete adjuvant, in order to induce Experimental allergic encephalomyelitis (EAE). About 0.5 mg of OX101, an anti-CD47 monoclonal antibody, was injected at day 10 and the disease progression of 5 animals followed (see Brostoff and Mason, (1984) J. Immunol. 133:1938-42). A control antibody of the same subclass (IgGl), OX21, was given to a further series of animals at the same time, and a further series was given phosphate-buffered saline (PBS).

The disease was monitored daily, and the disease scores averaged. The table below shows that there was a high incidence of the disease in the controls (PBS and OX21) but the disease was delayed and significantly less severe in the OX101 treated animals.

| | | | Days post MBP |
|---|---|---|---|
| PBS | OX21 | OX101 | injection |
| 0 | 0 | 0 | 10 |
| 0 | 0.2 | 0 | 11 |
| 0.2 | 0.6 | 0 | 12 |
| 1.2 | 1.2 | 0.1 | 13 |
| 1.5 | 2.4 | 1.2 | 14 |
| 2.5 | 2.4 | 1.5 | 15 |
| 1.6 | 0.9 | 1.7 | 16 |
| 0.2 | 0.1 | 0.3 | 17 |
| 0.2 | 0 | 0.2 | 18 |

1. Use of an agent capable of inhibiting the interaction between SIRP1α and CD47, in the preparation of a composition for the inhibition of macrophage involvement in autoimmune disease.

2. Use according to claim 1, wherein the agent is a ligand for CD47.

3. Use according to claim 1, wherein the agent is a ligand for SIRP1α.

4. Use according to claim 2 or claim 3, wherein the agent is an antibody.

5. Use according to claim 4, wherein the antibody is OX101.

6. Use according to any preceding claim wherein the autoimmune disease is selected from the group consisting of arthritis, inflammatory bowel disease and multiple sclerosis.

7. A method for the treatment of an autoimmune disease with macrophage involvement, comprising administering to a mammal an effective amount of an agent which inhibits the interaction between CD47 and SIRP1α.

8. A method for identifying an agent capable of inhibiting the interaction between CD47 and SIRP1α, comprising the steps of exposing one or more test compounds to CD 47 and/or SIRP1α, and monitoring the ability of the test compound to inhibit their interaction.

9. A method according to claim 8, comprising the steps of:
(a) incubating SIRP1α or CD47 with the compound or compounds to be assessed;
(b) identifying those compounds which bind to SIRP1α or CD47; and
(c) assessing those compounds which bind to SIRP1α or CD47 for the ability to modulate the interaction between SIRP1α and CD47.

10. A method according to claim 9, in which steps (a) and (b) are performed in vitro.

11. A method according to claim 9, for identifying a substance capable of disrupting an interaction between (i) SIRP1α, and (ii) CD47, which method comprises:
(a) providing a signal regulatory protein (SIRP) or a homologue, variant or derivative thereof, or a derivative thereof, as a first component;
(b) providing a CD47 protein or a homologue, variant or derivative thereof, as a second component;
(c) contacting the two components with a substance to be tested under conditions that would permit the two components to interact in the absence of the substance; and
(d) determining whether the substance disrupts the interaction between the first and second components.

12. A method according to claim 9, in which step (c) is performed in vivo.

13. A method according to claim 12, wherein step (c) is performed in an animal model for an autoimmune disease.

14. A method according to claim 13, wherein the animal model is EAE.
